(19) United States
(12) Patent Application Publication
(10) Pub. No.: US 2003/0092162 A1
(43) Pub. Date: May 15, 2003

(54) Chimeric adenoviral vectors for targeted gene delivery

(76) Inventors: Srinivas Shankara, Shrewsbury, MA (US); Donna Armentano, Belmont, MA (US); Bruce L. Roberts, Southborough, MA (US)

Correspondence Address: GENZYME CORPORATION, LEGAL DEPARTMENT, 15 PLEASANT ST CONNECTOR, FRAMINGHAM, MA, 01701-9322, US

(21) Appl. No.: 10/319,074

(22) Filed: Dec. 13, 2002

Related U.S. Application Data
(63) Continuation of application No. 09/663,076, filed on Sep. 15, 2000.
Application No. 09/663,076 is a continuation of application No. PCT/US99/06101, filed on Mar. 19, 1999.
(60) Provisional application No. 60/078,881, filed on Mar. 20, 1998.

(51) Int. Cl. C12Q 1/70; C12N 7/00; C12N 15/861

(52) U.S. Cl. 435/235.1; 435/5; 435/456

(57) ABSTRACT

The present invention provides chimeric adenoviral vectors that preferentially infect a target mammalian cell. Also provided are methods of targeting gene delivery to a specific cell type, treatment of cancer and methods of inducing a specific immune response in a subject. Pharmaceutical compositions are also provided.

### CROSS-REFERENCE TO RELATED APPLICATIONS

This application claims the priority under 35 U.S.C. §119(e) to U.S. Provisional Patent Application No. 60/078,881, filed Mar. 20, 1998. The priority application is hereby incorporated herein by reference in its entirety.

### TECHNICAL FIELD OF THE INVENTION

The present invention is directed to novel adenoviral vector systems that offer enhanced efficiency and specificity for gene delivery.

### BACKGROUND OF THE INVENTION

Searching for improved methods of intracellular delivery of therapeutically or biologically active molecules is of great medical importance. Gene transfer is generally defined as an approach for introducing an expressible polynucleotide (for example, a gene, a cDNA, or an mRNA patterned thereon) into a target cell. In a particular application of this approach, successful expression of an encoding polynucleotide leads to production in the target cell of a normal protein and leads to correction of a disease state associated with an abnormal gene. Therapies based on providing such proteins directly to target cells (protein replacement therapy) have generally been ineffective since, for example, the cell membrane presents a selectively permeable barrier to entry. Thus there is great interest in alternative methods to cause delivery of therapeutic proteins, especially by transfer of the relevant polynucleotide, often referred to as a transgene.

Viral vectors have been used with increasing frequency to date to deliver transgenes to target cells. Most attempts to use viral vectors for gene therapy have relied on retrovirus-based vectors, mainly because of their ability to integrate into the cellular genome. However, the disadvantages of retroviral vectors are becoming increasingly clear, including their tropism for dividing cells only, the possibility of insertional mutagenesis upon integration into the host genome, decreased expression of the transgene over time, rapid inactivation by serum complement, and the possibility of generating replication-competent retroviruses. See, for example, Jolly et al. (1994) Cancer Gene Therapy 1:51-64; and Hodgson et al. (1995) Bio Technology 13:222-5. Such disadvantages have led to the development of other viral-based vector systems, including those derived from adenoviruses.

Adenovirus is a DNA virus with a genome of about 36 kb that has been well-characterized through studies in classical genetics and molecular biology. Detailed discussion of adenovirus can be found in, for example, Shenk, “Adenoviridae and their Replication”, and M. S. Horwitz, “Adenoviruses”, Chapters 67 and 68, respectively, in VIROLOGY (B. N. Fields et al. eds.(1996)).

Adenoviruses are nonenveloped, regular icosahedrons (having 20 triangular surfaces and 12 vertices) that are about 65-80 nm in diameter. The capsid is composed of 252 subunits (capsomeres), of which 240 are hexons, and 12 are pentons. Each penton comprises a penton base on the surface of the capsid and a fiber protein projecting from the base. The fiber protein is itself generally composed of 3 identical polypeptide chains, the length thereof varies between serotypes.

It is understood that adenovirus utilizes two cellular receptors to attach to and infect a target cell. The fiber protein first attaches to a receptor on the surface of the target cell, and then penton base attaches to a further receptor, often a protein of the alpha integrin family. It has been determined that alpha-integrins often recognize short amino acid sequences on other cellular proteins for attachment purposes, including the tripeptide sequence Arg-Gly-Asp (abbreviated RGD). An RGD sequence is also found in the penton base protein of adenovirus and is currently understood in the art to mediate attachment of Ad to alpha integrins.

The human adenoviruses are divided into numerous serotypes (approximately 47, numbered accordingly) that are further classified into 6 subgroups, A through F, based upon properties including, but not limited to, hemagglutination of red blood cells, oncogenicity, DNA and amino acid sequence homology, and antigenicity. For example, the fiber protein, together with the hexon, are proposed to be the main determinants of the antigenicity and serotype specificity of adenovirus.

Because the infectious capabilities of the virus are closely associated with the interactions between surface proteins of the virus with the target cell, the serotype can serve as an important indicator for virus infection specificity of particular target cells. Little is known about the infection specificity and preference of different serotypes or serotype subgroups as to particular cell populations, including various cancer cells or dendritic cells. Most adenoviral vectors constructed so far use adenovirus serotypes from the well-studied subgroup C adenoviruses, especially Ad 2 and Ad 5 (Von Seggern et al. (1999) J. Virol. 73:1601-1608. Recently, other subgroup serotypes have been studied for their infectious properties that may shed a light on further design of improved adenoviral vectors. For example, the PCT publication WO96/26281 describes construction of a chimeric fiber protein in which the native Ad5 receptor binding domain is replaced with a normative Ad7 receptor binding domain, and shows that the replacement did not impair the ability of the virus to infect cells. However, no enhanced tropism of the constructed virus was shown as to the infected cells. U.S. Pat. No. 5,877,011 describes enhanced tropism for human airway epithelial cells by some of the adenoviral serotypes from subgroup D.

It is widely hoped that gene therapy will provide a long lasting and predictable form of therapy for certain disease states, such as cancers, cardiovascular diseases and some inherited diseases. Although adenoviral vectors are currently in clinical use and have shown therapeutic promise, a need remains to improve the infection efficiency of these vectors as to specific target cells in order to further improve their gene transfer capabilities. The present invention addresses this goal.

### DISCLOSURE OF THE INVENTION

The present invention relates to improved adenoviral vector systems that offer enhanced infection efficiency and delivery into preferred target cells of one or more therapeutically useful transgenes.

In some aspects of embodiments, the invention provides chimeric adenoviral vectors that comprise adenoviral nucleotide sequences of different serotypes and confer enhanced tropism for specific mammalian cells such as dendritic cells and cancer cells. The chimeric vectors may further comprise one or more transgenes coding for therapeutically useful proteins and therefore can be used as gene transfer vehicles in gene therapy applications. The invention also provides methods of providing an effective amount of a therapeutically or biologically active protein to target cells in a subject by administering to the subject the chimeric adenoviral vectors comprising a transgene encoding the protein, under conditions whereby the protein is expressed and activated to produce therapeutic or biological benefits in the subject.

In other aspects of embodiments, the invention provides recombinant adenoviruses comprising structural and functional protein components derived from adenoviral genomes of different serotypes serotype subgroups, wherein the viruses adopt preferred infection efficiency to specific target cells such as cancer cells or dendritic cells. The cell binding determinants, such as proteins from fiber, penton or hexon component, can be from an adenovirus serotype having preferred infection efficiency, and be provided in trans into the recombinant adenoviruses by a packaging cell line that express the cell binding determinants Von Seggern et al. (1998) J. Gen. Virol. 79:1461-1468. Alternatively, the recombinant adenoviruses can be produced by expressing the chimeric adenoviral vectors of the invention. The invention also provides methods of providing an effective amount of a therapeutically or biologically active protein to target cells in a subject by administering to the subject the recombinant adenoviruses comprising the protein, under conditions whereby the protein is presented and activated to produce therapeutic or biological benefits in the subject.

The invention also contemplates a method of identifying the infection efficiency of a known viral serotype to a specific cell population. Furthermore, the invention provides a method of characterizing an unknown cell type by determining the adenoviral subgroup(s) that preferentially infects the unknown cell, and comparing the infection profile to that of known cell types.

### BRIEF DESCRIPTION OF THE DRAWINGS

FIG. 1 depicts infection of melanoma cell lines by various adenovirus subgroups.

FIG. 2 depicts infection of colon cancer cells by various adenovirus subgroups.

FIG. 3 depicts infection of various cancer cells (breast, ovarian, cervical and prostate) by various adenovirus subgroups.

FIG. 4 depicts infection of human dendritic cells by various adenovirus subgroups.

FIG. 5 depicts a schematic map of various chimeric adenoviral vectors.

FIG. 6 depicts infection of human dendritic cells by chimeric adenoviral vectors.

### MODES FOR CARRYING OUT THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

The present invention is based on the recognition that adenovirus serotypes of different subgroups can preferentially infect specific target cells. By determining which serotype or subgroup of serotypes has enhanced infection efficiency to a particular target cell/tissue, various chimeric adenoviral vectors or recombinant adenoviruses can be generated to achieve enhanced targeted gene delivery of one or more genes to specific mammalian target cells such as tumor cells or antigen presenting cells.

The present invention provides, among others, a chimeric adenoviral vector comprising the genome of a first adenovirus as backbone, wherein the nucleotide sequence encoding a protein that facilitates binding of the first adenovirus to a target mammalian cell is replaced by the corresponding nucleotide sequence from a second adenovirus, wherein the second adenovirus belongs to a serotype that is not the same as the first adenovirus. In a representative aspect thereof, the replaced encoding sequence corresponds to the gene encoding the Ad fiber, hexon or penton base proteins, or combinations thereof.

In one embodiment of the invention, the nucleotide sequence encoding the entire fiber protein of the first adenovirus is replaced by that of the second adenovirus that is of different serotype subgroup. For example, a chimeric adenoviral vector of the invention comprises the genome of Ad2, a subgroup C serotype, as the backbone, within which the fiber protein coding sequence is removed and replaced with the fiber protein coding sequence of Ad17, a subgroup D serotype. The resulting chimeric adenoviral vector confers the targeting specificity of Ad17, which is different from that of Ad2.

The invention also provides a chimeric adenoviral vector comprising the genome of a first adenovirus as backbone, wherein a portion of the nucleotide sequence encoding a protein that facilitates binding of said vector to a target mammalian cell, or internalization thereof within said cell, is replaced by a portion of the corresponding gene from a second adenovirus, wherein the second adenovirus belongs to a different serotype subgroup. In a representative aspect thereof, the replaced encoding sequence corresponds to the gene encoding the Ad fiber, hexon or penton base proteins, or combinations thereof. Where a portion of the encoding sequence from a second adenovirus is used to construct a chimeric adenoviral vector, such sequence will have a length sufficient to confer a desired serotype-specific virus-cell interaction to the vector.

In one aspect, the portion of the nucleotide sequence being replaced is a portion of the fiber gene. The fiber protein monomer consists of a tail, a shaft, and a knob. Therefore, more preferably, the portion of the fiber gene being replaced can be anyone of these subunits and combinations thereof. According to one embodiment of the invention, a chimeric vector can be generated that comprises the tail of Ad2, and the shaft and knob of Ad 17. Another chimeric vector of the invention comprises the tail of Ad2, shaft of Ad17 and knob of Ad2. Still another chimeric vector of the invention comprises the tail and shaft of Ad2 and knob of Ad17. Other combinations of the fiber subunints of different serotypes are also encompassed by the invention.

Depending on the serotype of the second adenovirus nucleotide sequence used therein, a particular chimeric vector of the invention can more efficiently infect certain target cell. Therefore, one important aspect of the invention is the determination of the infection efficiency of representatives from each adenovirus subgroup as to various target cells. In one enbodiment of the invention, various adenovirus subgroups are identified that specifically or more efficiently infect certain mammalian target cells, such as dendritic cells or various cancer cells. It is discovered by the present inventor, for example, that serotypes Ad17 and Ad19 infect dendritic cells at high efficiency; serotypes Ad2, Ad31 and Ad41 can more effectively target melanoma cells; serotypes Ad2, Ad3, Ad4 and Ad17 target preferably various forms of colon cancer cells. Breast cancer cells can be specifically targeted by chimeric vectors having Ad2, Ad4 or Ad17 sequences; cervical cancer cells are targeted best by Ad17 and prostate cancer cells are targeted by Ad2 or Ad4.

In another embodiment of the invention, the chimeric adenoviral vectors further comprise a transgene. The transgene can be operably linked to a eukaryotic promoter to allow for expression therefrom in the target mammalian cell. The transgene can be a cytotoxic agent, a tumor antigen, a costimulatory molecule or a cytokine. For example, when the chimeric vector is targeted to dendritic cells that are to be used in cancer immunotherapy, the transgene may encode autologous or allogeneic tumor-associated antigens. When these antigens are expressed and presented on the surface of the dendritic cells administered into a subject, they are capable of eliciting an tumor-specific immune response in the subject by activating a population of cytotoxic T lymphocytes (CTLs). The transgenes also can encode ribozymes or antisense molecules or other regulatory polynucleotides.

Additional aspects of the invention include methods to provide biologically active and/or therapeutic proteins to a mammalian cell, including but not limited to, a neoplastic cell, in order to provide therapeutic benefit. According to this aspect of the invention, the chimeric adenoviral vectors typically include a transgene encoding a prophylactic or therapeutic product which is expressed by the vector in the target cell.

A still further representative aspect of the invention involves providing a biologically active and/or therapeutic protein to a patient by administering dendritic cells transduced with a chimeric adenoviral vector. Preferably, the vector comprises the fiber elements of a subgroup D serotype, and a transgene encoding the protein that is operably linked to a eukaryotic promoter to allow for expression therefrom in a mammalian cell, under conditions whereby the biologically active and/or therapeutic protein is expressed, and the desired phenotypic benefit is produced in said subject.

In other aspects of embodiments, the invention provides recombinant adenoviruses comprising structural and functional protein components derived from adenoviral genomes of different serotypes serotype subgroups, wherein the viruses adopt preferred infection efficiency to specific target cells such as cancer cells or dendritic cells The cell binding determinants, such as proteins from fiber, penton or bexon component, can be from an adenovirus serotype having preferred infection efficiency, and be provided in trans into the recombinant adenoviruses by a packaging cell line that express the cell binding determinants. Von Seggern et al. (1998) J. Gen. Virol. 79:1461-1468. Alternatively, the recombinant adenoviruses can be produced by expressing the chimeric adenoviral vectors of the invention. The invention also provides methods of providing an effective amount of a therapeutically or biologically active protein to target cells in a subject by administering to the subject the recombinant adenoviruses comprising the protein, under conditions whereby the protein is presented and activated to produce therapeutic or biological benefits in the subject.

The invention also contemplates a method of identifying the infection efficiency of a known viral serotype to a specific cell population. Furthermore, the invention provides a method of characterizing an unknown cell type by determining the adenoviral subgroup(s) that preferentially infects the unknown cell, and comparing the infection profile to that of known cell types. For example, an unidentified cancer cell that is preferentially infected by subgroups A, C and F, may be potentially identified as a melanoma-like cell. Similarly, an unidentified cell that is preferentially infected by subgroup D may be analogous to a dendritic cell.

The practice of the present invention will employ, unless otherwise indicated, conventional techniques of molecular biology, microbiology, cell biology and recombinant DNA, which are within the skill of the art. See, e.g., Sambrook et al., MOLECULAR CLONING: A LABORATORY MANUAL, 2 d edition (1989); CURRENT PROTOCOLS IN MOLECULAR BIOLOGY (Ausubel et al. eds. (1987)); the series METHODS IN ENZYMOLOGY (Academic Press, Inc.); ANTIBODIES: A LABORATORY MANUAL (E. Harlow and D. Lane eds. (1988)); PCR 2: A PRACTICAL APPROACH (M. J. MacPherson et al. eds. (1995)) and ANIMAL CELL CULTURE (Freshney ed. (1987)).

Definitions

As used herein, certain terms may have the following defined meanings.

As used in the specification and claims, the singular forms “a”, “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

The terms “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-, double-stranded and triple helical molecules, a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A nucleic acid molecule may also comprise modified nucleic acid molecules.

A “gene” is a hereditary unit that, in the classical sense, occupies a specific position (locus) within the genome or chromosome; a unit that has one or more specific effects upon the phenotype of the organism; a unit that can mutate to various allelic forms; a unit that recombines with other such units. Three classes of genes are now recognized: (1) structural genes that are transcribed into mRNAs, which are then translated into polypeptide chains, (2) structural genes that are transcribed into rRNA or tRNA molecules which are used directly, and (3) regulatory genes that are not transcribed, but serve as recognition sites for enzymes and other proteins involved in DNA replication and transcription.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid” refers to either natural and/or unnatural or synthetic amino acids, including glycine and both the D or L optical isomers, and amino acid analogs and peptidomimetics. A peptide of three or more amino acids is commonly called an “oligopeptide.” If the peptide chain is long, the peptide is commonly called a “polypeptide” or a “protein.”

A “transgene” is the term given the to the polynucleotide carried by the gene delivery vehicle. A “gene delivery vehicle” is defined as any molecule that can carry inserted polynucleotides into a host cell. Examples of other gene delivery vehicles include liposomes, viruses, such as baculovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression. The term “transduction” refers to the transfer of polynucleotides into a host cell.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors and the like.

Polynucleotides are inserted into vector genomes using methods well known in the art. For example, insert and vector DNA can be contacted, under suitable conditions, with a restriction enzyme to create complementary ends on each molecule that can pair with each other and be joined together with a ligase. Alternatively, synthetic nucleic acid linkers can be ligated to the termini of restricted polynucleotide. These synthetic linkers contain nucleic acid sequences that correspond to a particular restriction site in the vector DNA. Additionally, an oligonucleotide containing a termination codon and an appropriate restriction site can be ligated for insertion into a vector containing, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; versatile multiple cloning sites; and T7 and SP6 RNA promoters for in vitro transcription of sense and antisense RNA. Other means are well known and available in the art.

An “adenoviral vector” is defined as a recombinantly produced adenovirus or viral particle that comprises a polynucleotide to be delivered into a host cell, either in vivo, ex vivo or in vitro. A “chimeric adenoviral vector” is an adenoviral vector that comprises polynucleotides from more than one adenovirus serotype. For example, a chimeric adenoviral vector may contain polynucleotides encoding the backbone of one subgroup serotype and other proteins (e.g., fiber or other structural proteins) from one or more different subgroup serotypes.

Viral “packaging” as used herein refers to a series of intracellular events that results in the synthesis and assembly of a viral vector. Packaging typically involves the replication of the “pro-viral genome”, or a recombinant pro-vector typically referred to as a “vector plasmid” (which is a recombinant polynucleotide than can be packaged in an manner analogous to a viral genome, typically as a result of being flanked by appropriate viral “packaging sequences”), followed by encapsidation or other coating of the nucleic acid. Thus, when a suitable vector plasmid is introduced into a packaging cell line under appropriate conditions, it can be replicated and assembled into a viral particle. Viral “rep” and “cap” genes, found in many viral genomes, are genes encoding replication and encapsidation proteins, respectively.

A “replication-deficient” or “replication-incompetent” viral vector refers to a viral vector in which one or more functions necessary for replication and/or packaging are missing or altered, rendering the viral vector incapable of initiating viral replication following uptake by a host cell. To produce stocks of such replication-defective viral vectors, the virus or pro-viral nucleic acid can be introduced into a “packaging cell line” that has been modified to contain genes encoding the missing functions which can be supplied in trans). For example, such packaging genes can be stably integrated into a replicon of the packaging cell line or they can be introduced by transfection with a “packaging plasmid” or helper virus carrying genes encoding the missing functions.

“Target cell” is intended to include any individual cell or cell culture which can be or have been recipients for vectors or the incorporation of exogenous nucleic acid molecules, polynucleotides and/or proteins. It also is intended to include progeny of a single cell, and the progeny may not necessarily be completely identical (in morphology or in genomic or total DNA complement) to the original parent cell due to natural, accidental, or deliberate mutation The cells may be prokaryotic or eukaryotic, and include but are not limited to bacterial cells, yeast cells, animal cells, and mammalian cells, e.g., murine, rat, simian or human cells.

The terms “cancer,” “neoplasm,” and “tumor,” used interchangeably and in either the singular or plural form, refer to a cell population that has undergone a malignant transformation that makes them pathological to the host organism. Primary cancer cells (that is, cells obtained from near the site of malignant transformation) can be readily distinguished from non-cancerous cells by well-established techniques, particularly histological examination.

The terms “tumor cell”, “neoplastic cell”, or “cancer cell”, used either in the singular or plural form, refer to proliferating cells that have undergone a malignant transformation that makes them pathological to the host organism. A neoplastic cell is said to be benign if it does not undergo metastasis and malignant if it undergoes metastasis. A metastatic cell means that the cell can invade and destroy neighboring body structures. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g. by such procedures as CAT scan, magnetic resonance imaging (MRI), X-ray, ultrasound or palpation. Biochemical or immunologic findings alone may be insufficient to meet this definition. Tumor cells often express antigens which are tumor specific. The term “tumor associated antigen” or “TAA” refers to an antigen that is associated with or specific to a tumor.

“Dendritic cells” or “DCs” are potent antigen-presenting cells (APCs) in that they are capable of inducing the presentation of one or more antigens, preferably in association with class 1, but not class II MHC molecules. It has been shown that DCs provide all the signals required for T cell activation and proliferation. These signals can be categorized into two types. The first type, which gives specificity to the immune response, is mediated through interaction between the T-cell receptor/CD3 (“TCR/CD3”) complex and an antigenic peptide presented by a major histocompatibility complex (“MHC”) class I or II protein on the surface of APCs. This interaction is necessary, but not sufficient, for T cell activation to occur. In fact, without the second type of signals, the first type of signals can result in T cell anergy. The second type of signals, called costimulatory signals, is neither antigen-specific nor MHC-restricted, and can lead to a full proliferation response of T cells and induction of T cell effector functions in the presence of the first type of signals. Various methods for generating dendritic cells from peripheral blood or bone marrow progenitors have been described, see, for example, Inaba et al. (1992) J. Exp. Med. 175:1157; Inaba et al. (1992) J. Exp, Med. 176:1693-1702; Romani et al. (1994) J. Exp. Med. 180:83-93; Sallusto et al. (1994) J. Exp. Med. 179:1109-18; Bender et al. (1996) J. Imm. Methods 196:121-135; and Romani et al. (1996) J. Imm. Methods 196:137-151.

“Co-stimulatory molecules” are involved in the interaction between receptor-ligand pairs expressed on the surface of antigen presenting cells and T cells. Research accumulated over the past several years has demonstrated convincingly that resting T cells require at least two signals for induction of cytokine gene expression and proliferation (Schwartz (1990) Science 248:1349-1356; Jenkins (1992) Immunol. Today 13:69-73). One signal, the one that confers specificity, can be produced by interaction of the TCR/CD3 complex with an appropriate MHC/peptide complex. The second signal is not antigen specific and is termed the “co-stimulatory” signal. This signal was originally defined as an activity provided by bone-marrow-derived accessory cells such as macrophages and dendritic cells, the so called “professional” APCs. Several molecules have been shown to enhance co-stimulatory activity. These are heat stable antigen (HSA) (Liu et al. (1992) J. Exp. Med. 175:437445), chondroitin sulfate-modified MHC invariant chain (Ii-CS) (Naujokas et al. (1993) Cell 74:257-268), intracellular adhesion molecule I (ICAM-1) (Van Seventer (1990) J. Immunol. 144:45794586), B7-1, and B7-2/B70 (Schwartz (1992) Cell 71:1065-1068). These molecules each appear to assist co-stimulation by interacting with their cognate ligands on the T cells. Co-stimulatory molecules mediate co-stimulatory signal(s) which are necessary, under normal physiological conditions, to achieve full activation of naïve T cells. One exemplary receptor-ligand pair is the B7 costimulatory molecule on the surface of APCs and its counter-receptor CD28 or CTLA-4 on T cells (Freeman et al. (1993) Science 262:909-911; Young et al. (1992) J. Clin. Invest. 90: 229; Nabavi et al. (1992) Nature 360:266-268). Other important co-stimulatory molecules are CD40, CD54, CD80, CD86. The term “co-stimulatory molecule” encompasses any single molecule or combination of molecules which, when acting together with a peptide/MHC complex bound by a TCR on the surface of a T cell, provides a co-stimulatory effect which achieves activation of the T cell that binds the peptide. The term thus encompasses B7, or other co-stimulatory molecule(s) on an antigen-presenting matrix such as an APC, fragments thereof (alone, complexed with another molecule(s), or as part of a fusion protein) which, together with peptide/MHC complex, binds to a cognate ligand and results in activation of the T cell when the TCR on the surface of the T cell specifically binds the peptide. Co-stimulatory molecules are commercially available from a variety of sources, including, for example, Beckman Coulter. It is intended, although not always explicitly stated, that molecules having similar-biological activity as wild-type or purified co-stimulatory molecules (e.g., recombinantly produced or muteins thereof) are intended to be used within the spirit and scope of the invention.

“Gene delivery,” “gene transfer,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

“In vivo” gene delivery, gene transfer, gene therapy and the like as used herein, are terms referring to the introduction of a vector comprising an exogenous polynucleotide directly into the body of an organism, such as a human or non-human mammal, whereby the exogenous polynucleotide is introduced to a cell of such organism in vivo.

A “subject” is a vertebrate, preferably a mammal, more preferably a human. Mammals include, but are not limited to, murines, simians, humans, farm animals, sport animals, and pets.

An “effective amount” is an amount sufficient to effect beneficial or desired results. An effective amount can be administered in one or more administrations, applications or dosages. For purposes of this invention, an effective amount of hybrid cells is that amount which promotes expansion of the antigenic-specific immune effector cells, e.g., T cells.

The term “cytokine” refers to any immunomodulatory factor that exerts a variety of effects on cells, for example, inducing growth or proliferation. Non-limiting examples of cytokines include, IL-2, stem cell factor (SCF), IL-3, IL-6, IL-12, G-CSF, GM-CSF, IL-1α, IL-11, MIP-1α, LIF, c-kit ligand, TPO, and flt3 ligand. Cytokines are commercially available from several vendors such as, for example, Genentech (South San Francisco, Calif.), Amgen (Thousand Oaks, Calif.) and Immunex (Seattle, Wash.). It is intended, although not always explicitly stated, that molecules having similar biological activity as wild-type or purified cytokines (e.g., recombinantly produced) can also be used within the spirit and scope of the invention.

A “composition” is intended to mean a combination of active agent and another compound or composition, inert (for example, a detectable agent or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

Materials and Methods

Identification of Cell Targeting Preferences of Adenovirus Serotypes

Identification of adenovirus subgroups that best infects a particular cell type is useful in designing vectors to deliver genes to that cell type for in vivo gene therapy. Infectivity can be determined by any method known in the art. For example, the target cells can be incubated with various adenovirus serotypes and the total number of cells measured by DAPI staining. The percentage of infected cells can be determined by staining the cells with an anti-hexon protein antibody. Using this strategy, for example, the present inventors have identified adenovirus subgroups that efficiently and specifically infect dendritic cells and various cancer cells. These results can be used in designing and generating chimeric vectors specific for cancer cells or dendritic cells.

Once the specific cell targets of one or more adenoviral subgroups has been determined, the present invention can also be used to identify unknown cells. For example, an unidentified cancer cell that is preferentially infected by subgroups A, C and F, may be potentially identified as a melanoma-like cell. Similarly, an unidentified cell that is preferentially infected by subgroup D may be a dendritic cell.

Construction of Chimeric Adenoviral Vectors

The present invention also provides chimeric adenoviral vectors which are targeted to specific cell types. In order to construct the chimeric adenoviral vectors of the invention, reference may be made to the substantial body of literature on how such vectors may be designed, constructed and propagated using techniques from molecular biology and microbiology that are well-known to the skilled artisan. Construction of the chimeric adenoviral vectors can be based on adenovirus DNA sequence information widely available in the field, e.g., nucleic acid sequence published in databases such as GenBank.

It is known within the state of the art that minor modification to a nucleotide sequence will not affect the function of the molecules encoded thereby. Thus, biologically equivalent polynucleotides and genomes of published sequences are also useful in the methods described herein. These polynucleotides can be identified by hybridization under stringent conditions to the sequences disclosed in the published references or known in the art. Alternatively, the polynucleotides can be identified as being at least 80%, or more preferably, at least 90% or most preferably, at least 95%, identical to the disclosed sequences using sequence alignment programs and default parameters.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions of about 6×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

That a polynucleotide or polynucleotide region (or a polypeptide or polypeptide region) has a certain percentage (for example, 80%, 85%, 90%, or 95%) of “sequence identity” to another sequence means that, when aligned, that percentage of bases (or amino acids) are the same in comparing the two sequences. This alignment and the percent homology or sequence identity can be determined using software programs known in the art, for example those described in CURRENT PROTOCOLS IN MOLECULAR BIOLOGY (F. M. Ausubel et al. eds. (1987)) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code standard; filter=none; strand=both; cutoff=60; expect=10; Matrix BLOSUM62; Descriptions=50 sequences; sort by HIGH SCORE; Databases non-redundant, GenBank+EMBL+DDBJ+PDB+GenBank CDS translations+SwissProtein+SPupdate+PIR. Details of these programs can be found at the following Internet address: http://www.ncbi.nlmn.nih.gov/cgi-bin/BLAST.

In one aspect of the invention, the adenoviral genome used to construct the chimeric vector is derived from a replication-deficient adenovirus. Using replication-deficient adenovirus genome can avoid viral over-reproduction inside the cell and transfer to other cells and infection of other people, which means the viral infection activity is effectively limited to the first infected target cell. Specific examples of adenoviral vector genomes which can be used as the backbone for a chimeric adenoviral vector of the invention include, for example, Ad2/CFTR-1 and Ad2/CFTR-2 and others described in U.S. Pat. No. 5,670,488. Such vectors may include deletion of the El region, partial or complete deletion of the E4 region, and deletions within, for example, the E2 and E3 regions. Within the scope of the invention are, for example, chimeric vectors which contain an Ad2 backbone with one or more target cell-specific Ad serotype fiber proteins or fragments thereof in the virus. Other adenoviral vector genomic designs which can be used in the chimeric adenoviral vectors of the invention include those derived from U.S. Pat. Nos. 5,707,618 and 5,824,544.

To construct the recombinant chimeric adenoviral vectors of the invention which contain a transcription unit, the skilled artisan can use standard techniques of molecular biology to engineer a transgene or a capsid protein into a backbone vector genome (Berkner (1992) Curr. Top. Micro. Immunol. 158:39-66). For example, a plasmid containing a transgene and any operably linked regulatory elements inserted into an adenovirus genomic fragment can be co-transfected with a linearized viral genome derived from an adenoviral vector of interest into a recipient cell under conditions whereby homologous recombination occurs between the genomic fragment and the virus. Preferably, a transgene is engineered into the site of an El deletion. As a result, the transgene is inserted into the adenoviral genome at the site in which it was cloned into the plasmid, creating a recombinant adenoviral vector. The chimeric adenoviral vectors can also be constructed using standard ligation techniques, for example, removing a restriction fragment containing a fiber gene or portion thereof from a first adenovirus and ligating into that site a restriction fragment containing the corresponding fiber gene or portion thereof from a second adenovirus. A representative example of a chimeric adenoviral vector of the invention is Ad 2/βgal-2 fiber 17 (exemplified below in Example 3).

Preparation of replication-deficient chimeric adenoviral vector stocks can be accomplished using cell lines that complement viral genes deleted from the vector, e.g., 293 or A549 (available from the ATCC) cells containing the deleted adenovirus El genomic sequences. After amplification of plaques in suitable complementing cell lines, the viruses can be recovered by freeze-thawing and subsequently purified using cesium chloride centrifugation. Alternatively, virus purification can be performed using chromatographic techniques that are well known in the art.

Titers of replication-deficient chimeric adenoviral vector stocks can be determined by plaque formation in a complementing cell line, e.g., 293 cells. Endpoint dilution using an antibody to the adenoviral hexon protein may be used to quantitate virus production or infection efficiency of target cells (Armentano et al. (1995) Hum. Gene Ther. 6:1343-53.) Transgenes which can be delivered and expressed from a chimeric adenoviral vector of the invention include, but are not limited to, those encoding enzymes, blood derivatives, hormones, lymphokines such as the interleukins and interferons, coagulants, growth factors, neurotransmitters, tumor suppressors, apoliproteins, antigens, and antibodies, and other biologically active proteins. Specific transgenes which may be encoded by the chimeric adenoviral vectors of the invention include, but are not limited to, genes encoding tumor antigens, e.g., MART-1, Gp100, NY-ESO-1, GA-733, HER-2/neu; genes encoding costimulatory molecules, e.g., B7.1, B7.2, CD40L, HLA Class I and II; cytotoxic genes, e.g., herpes simplex virus thymidine kinase (HSV-tk), cytosine deaminase; immunomodulatory molecules, e.g., costimulatory molecules, cytokines (GM-CSF, IL-2, etc.), heat shock proteins and the like. Transgenes encoding antisense molecules or ribozymes are also within the scope of the invention. The vectors may contain one or more transgenes under the control of one or more regulatory elements.

In addition to containing the DNA sequences encoding one or more transgenes, the chimeric adenoviral vectors of the invention may contain any expression control sequences such as a promoter or enhancer, a polyadenylation element, and any other regulatory elements that may be used to modulate or increase expression, all of which are operably linked in order to allow expression of the transgene. The use of any expression control sequences, or regulatory elements, which facilitate expression of the transgene is within the scope of the invention. Such sequences or elements may be capable of generating tissue-specific expression or be susceptible to induction by exogenous agents or stimuli.

Preparation of Dendritic Cells

Dendritic cells are specialized antigen presenting cells (APCs) that are critical for eliciting T cell mediated immune responses. At least two methods have been used for the generation of human dendritic cells from hematopoietic precusor cells in peripheral blood. One approach utilizes the rare CD34+ precursor cells and stimulate them with GM-CSF plus TNF-α. The other method makes use of the more abundant CD34− precursor population and stimulate them with GM-CSF plus IL-4.

In one aspect of the invention, the method described in Romani et al (1996), supra; and Bender et al (1996), supra is used to generate both immature and mature dendritic cells from the peripheral blood mononuclear cells (PBMC) of a mammal, such as a murine, simian or human. Briefly, isolated PBMC are pre-treated to deplete T- and B-cells by means of an immunomagnetic technique. Lymphocyte-depleted PBMC are then cultured for 7 days in RPMI medium, supplemented with 1% autologous human plasma and GM-CSF/IL-4, to generate dendritic cells. Dendritic cells are nonadherent when compared to their monocyte progenitors. Thus, on day 7, non-adherent cells are harvested for further processing.

The dendritic cells derived from PBMC in the presence of GM-CSF and IL-4 are immature, in that they can lost the nonadherence property and revert back to macrophage cell fate if the cytokine stimuli are removed from the culture. The dendritic cells in an immature state are very effective in processing native protein antigens for the MHC class II restricted pathway (Romani et al. (1989) J. Exp. Med. 169:1169.

Further maturation of cultured dendritic cells is accomplished by culturing for 3 days in a macrophage-conditioned medium (CM), which contains the necessary maturation factors. Mature dendritic cells are less able to capture new proteins for presentation but are much better at stimulating resting T cells (both CD4+ and CD8+) to grow and differentiate.

Mature dendritic cells can be identified by their change in morphology, such as the formation of more motile cytoplasmic processes; by their nonadherence; by the presence of at least one of the following markers: CD83, CD68, HLA-DR or CD86; or by the loss of Fc receptors such as CD115 (reviewed in Steinman (1991) Annu. Rev. Immunol. 9:271.

Mature dendritic cells can be collected and analyzed using typical cytofluorography and cell sorting techniques and devices, such as FACScan and FACStar. Primary antibodies used for flow cytometry are those specific to cell surface antigens of mature dendritic cells and are commercially available. Secondary antibodies can be biotinylated Igs followed by FITC- or PE-conjugated streptavidin.

Alternatively, others have reported that a method for upregulating (activating) dendritic cells and converting monocytes to an activated dendritic cell phenotype. This method involves the addition of calcium ionophore to the culture media convert monocytes into activated dendritic cells. Adding the calcium ionophore A23187, for example, at the beginning of a 2448 hour culture period resulted in uniform activation and dendritic cell phenotypic conversion of the pooled “monocyte plus DC” fractions: characteristically, the activated population becomes uniformly CD14 (Leu M3) negative, and upregulates HLA-DR, HLA-DQ, ICAM-1, B7.1, and B7.2. Furthermore this activated bulk population functions as well on a small numbers basis as a further purified.

Specific combination(s) of cytokines have been used successfully to amplify (or partially substitute) for the activation/conversion achieved with calcium ionophore: these cytokines include but are not limited to G-CSF, GM-CSF, IL-2, and IL-4. Each cytokine when given alone is inadequate for optimal upregulation.

Infection of Target Cells

The chimeric adenoviral vectors described herein can be used either alone or in a suitable carrier for the infection of target cells.

The infection efficiency of the chimeric adenoviral vectors of the invention may be assayed by standard techniques to determine the infection of target cells. Such methods include, but are not limited to, plaque formation, end-point dilution using, for example, an antibody to the adenoviral hexon protein, and cell binding assays using radiolabelled virus. Improved infection efficiency may be characterized as an increase in infection of at least an order of magnitude with reference to a control virus. Where a chimeric adenoviral vector encodes a marker or other transgene, relevant molecular assays to determine expression include the measurement of transgene mRNA, by, for example, Northern blot, S1 analysis or reverse transcription-polymerase chain reaction (RT-PCR). The presence of a protein encoded by a transgene may be detected by Western blot, immunoprecipitation, immunocytochemistry, CAT assay or other techniques known to those skilled in the art. Marker-specific assays can also be used, such as X-gal staining of cells infected with a chimeric adenoviral vector encoding β-galactosidase.

Infection may also be facilitated by the use of cationic molecules, such as cationic lipids as disclosed in PCT WO96/18372. Cationic amphiphiles which may be used to form complexes with the chimeric adenoviral vectors of the invention include, but are not limited to, cationic lipids, such as DOTMA (Felgner et al. (1987) PNAS USA 84:7413-17) (N-[1-(2,3-dioletloxy)propyl]-N,N,N-trimethylammonium chloride); DOGS (dioctadecylamidoglycylspermine) (Behr et al. (1989) PNAS USA 86:6982-86); DMRIE (1,2-dimyristyloxypropyl-3-dimethyl-hydroxyethyl ammonium bromide) (Felgner et al. (1994) J. Biol. Chem. 269:2550-61; and DC-chol (3B [N-N′,N′-dimethylaminoethane)-carbarnoyl] cholesterol) (U.S. Pat. No. 5,283,185). The use of other cationic amphiphiles recognized in the art or which come to be discovered is within the scope of the invention. In the formulation of complexes containing a cationic amphiphile with a chimeric adenoviral vector, a preferred range of 10⁷ 10¹⁰ infectious units of virus may be combined with a range of 10⁴-10⁶ cationic amphiphile molecules/viral particle.

Animal Models

In order to determine transgene expression and infection efficiency in vivo using the constructs and compositions of the invention, animal models may be particularly relevant in order to assess transgene persistence against a background of potential host immune response. Such a model may be chosen with reference to such parameters as case of delivery, identity of transgene, relevant molecular assays, and assessment of clinical status. Where the transgene encodes a protein whose lack is associated with a particular disease state, an animal model which is representative of the disease state may optimally be used in order to assess a specific phenotypic result and clinical improvement. However, it is also possible that particular chimeric adenoviral vectors of the invention display enhanced infection efficiency only in human model systems, e.g., using primary cell cultures, tissue explants, or permanent cell lines. In such circumstances where there is no animal model system available in which to model the infection efficiency of a chimeric adenoviral vector with respect to human cells, reference to art-recognized human cell culture models will be most relevant and definitive.

Relevant animals in which the chimeric adenoviral vectors may be assayed include, but are not limited to, mice, rats, monkeys, and rabbits. Suitable mouse strains in which the vectors may be tested include, but are not limited to, C3H, C57BI/6 (wild-type and nude) and Balb/c (available from Taconic Farms, Germantown, N.Y.).

Where it is desirable to assess the host immune response to vector administration, testing in immune-competent and immune-deficient animals may be compared in order to define specific adverse responses generated by the immune system. The use of immune-deficient animals, e.g., nude mice, may be used to characterize vector performance and persistence of transgene expression, independent of an acquired host response.

In vivo, Ex vivo and In vitro Use

The chimeric adenoviral vectors of the invention have a number of in vivo and in vitro utilities. The vectors can be used to transfer a normal copy of a transgene encoding a biologically active protein to target cells in order to remedy a deficient or dysfunctional protein. The vectors can be used to transfer marked transgenes (e.g., containing nucleotide alterations) which allow for distinguishing expression levels of a transduced gene from the levels of an endogenous gene. The chimeric adenoviral vectors can also be used to define the mechanism of specific viral protein-cellular protein interactions that are mediated by specific virus surface protein sequences. The vectors can also be used to optimize infection efficiency of specific target cells by adenoviral vectors, for example, using a chimeric adenoviral vector containing Ad 17 fiber protein to infect human dendritic cells. Where it is desirable to use an adenoviral vector for gene transfer to cancer cells in an individual, a chimeric adenoviral vector can be chosen which selectively infects the specific type of target cancer cell and avoids promiscuous infection. Where primary cells are isolated from a tumor in an individual requiring gene transfer, the cells may be tested against a panel of chimeric adenoviral vectors to select a vector with optimal infection efficiency for gene delivery. The vectors can further be used to transfer tumor antigens to dendritic cells which can then be delivered to an individual to elicit an anti-tumor immune response. Chimeric adenoviral vectors can also be used to evade undesirable immune responses to particular adenovirus serotypes which compromise the gene transfer capability of adenoviral vectors.

The present invention is further directed to compositions containing the chimeric adenoviral vectors of the invention which can be administered in an amount effective to deliver one or more desired transgenes to the cells of an individual in need of such molecules and cause expression of a transgene encoding a biologically active protein to achieve a specific phenotypic result. The cationic amphiphile-plasmid complexes or cationic amphiphile-virus complexes may be formulated into compositions for administration to an individual in need of the delivery of the transgenes.

The compositions can include physiologically acceptable carriers, including any relevant solvents. As used herein, “physiologically acceptable carrier” includes any and all solvents, dispersion media, coatings, antibacterial and antifungal agents, isotonic and absorption delaying agents, and the like. Except insofar as any conventional media or agent is incompatible with the active ingredient, its use in the compositions is contemplated.

Routes of administration for the compositions containing the chimeric adenoviral vectors of the invention include conventional and physiologically acceptable routes such as direct delivery to a target organ or tissue, intranasal, intravenous, intramuscular, subcutaneous, intradermal, oral and other parenteral routes of administration.

The invention is further directed to methods for using the compositions of the invention in vivo or ex vivo applications in which it is desirable to deliver one or more transgenes into cells such that the transgene produces a biologically active protein for a normal biological or phenotypic effect. In vivo applications involve the direct administration of one or more chimeric adenoviral vectors formulated into a composition to the cells of an individual. Ex vivo applications involve the transfer of a composition containing the chimeric adenoviral vectors directly to autologous cells which are maintained in vitro, followed by readministration of the transduced cells to a recipient.

Dosage of the chimeric adenoviral vector to be administered to an individual for expression of a transgene encoding a biologically active protein and to achieve a specific phenotypic result is determined with reference to various parameters, including the condition to be treated, the age, weight and clinical status of the individual, and the particular molecular defect requiring the provision of a biologically active protein. The dosage is preferably chosen so that administration causes a specific phenotypic result, as measured by molecular assays or clinical markers. For example, determination of the infection efficiency of a chimeric adenoviral vector containing a transgene which is administered to an individual can be performed by molecular assays including the measurement of mRNA of the transgenes product, by, for example, Northern blot, S1 or RT-PCR analysis or the measurement of the protein as detected by Western blot, immunoprecipitation, immunocytochemistry, or other techniques known to those skilled in the art. Relevant clinical studies which could be used to assess phenotypic results from delivery of the transgene include assessment of tissue function and radiological evaluation. Transgene expression in disease states can be assayed using the specific clinical parameters most relevant to the particular condition Dosages of a chimeric adenoviral vector which are effective to provide expression of a transgene encoding a biologically active protein and achieve a specific phenotypic result range from approximately 10⁸ infectious units (I.U.) to 10¹¹ I.U. for humans.

It is especially advantageous to formulate parenteral compositions in dosage unit form for ease of administration and uniformity of dosage. Dosage unit form as used herein refers to physically discrete units suited as unitary dosages for the subjects to be treated, each unit containing a predetermined quantity of active ingredient calculated to produce the specific phenotypic effect in association with the required physiologically acceptable carrier. The specification for the novel dosage unit forms of the invention are dictated by and directly depend on the unique characteristics of the chimeric adenoviral vector and the limitations inherent in the art of compounding. The principal active ingredient (the chimeric adenoviral vector) is compounded for convenient and effective administration in effective amounts with the physiologically acceptable carrier in dosage unit form as discussed above.

Maximum benefit and achievement of a specific phenotypic result from administration of the chimeric adenoviral vectors of the invention may require repeated administration. Such repeated administration may involve the use of the same chimeric adenoviral vector, or, alternatively, may involve the use of different chimeric adenoviral vectors which are rotated in order to alter viral antigen expression and decrease host immune response.

The invention is further illustrated by the following specific examples which are not intended in any way to limit the scope of the invention.

### EXAMPLES

### Example 1

### Infection of Cancer Cells With Different Adenovirus Serotypes

Infection efficiencies of representatives from each adenovirus subgroup on various cancer cells was determined. Melanoma (WM-115, A375 and SK-MEL-2 cell lines), colon (CaCO2, HCT-116 and HT-29 cell lines), breast (SK-BR-3 cell line), cervical (HeLa), prostate (PC-3 line) and ovarian (SK-OV3) cancer cell lines were obtained from the American Type Culture Collection (ATCC), Manassas, Va. The cells were plated at a density of 2×10⁴ cells/well in a 96 well plate. After 24 hours, the cells were infected at an moi of 5. Thirty-six hours post infection, the cells were fixed with 1:1 acetone:methanol, permeablized with a solution of 0.05% Tween 20 in PBS, and stained with FITC labeled anti-hexon antibody (Chemicon, Temecula, Calif.) to visualize cells that had been productively infected (i.e. to visualize virus replication). Cells were also subjected to the DAPI staining procedure in order to visualize the total number of nuclei. The results could be readily determined upon simple inspection. A549 cells were used as a control.

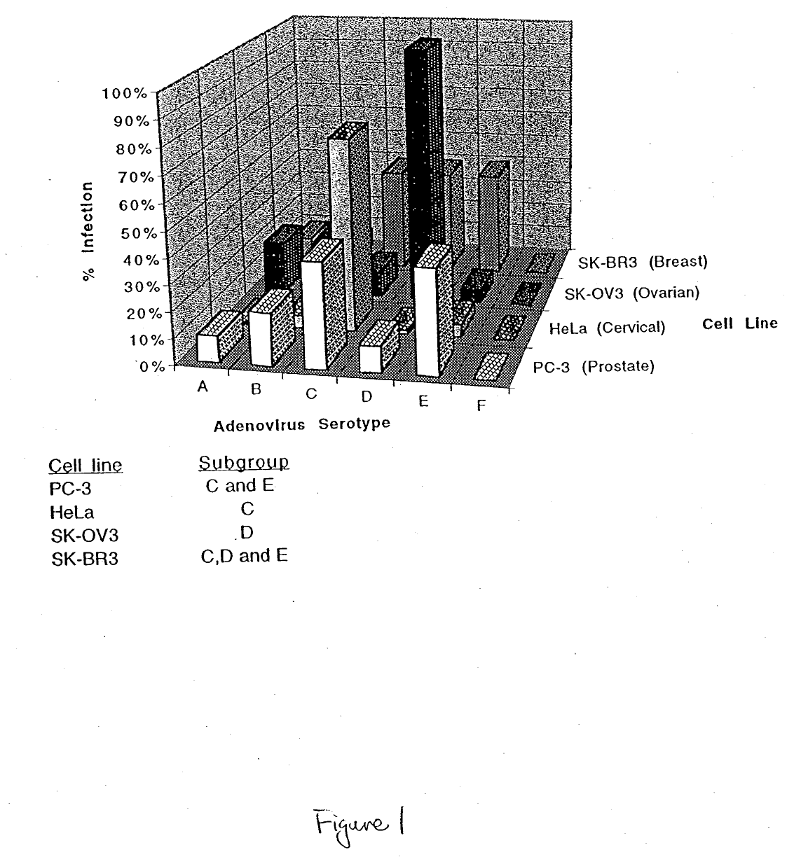

FIG. 1 shows infection efficiency of the various subgroups into the melanoma cell lines, SK-MEL2, A375 and WM115. Subgroup A (Ad 31) was most efficient at infecting A375 cells, while subgroups C (Ad 2) and F (Ad 41) were more efficient at infection of SK-MEL-2 cells. No subgroup was more efficient at infecting WM115 cells.

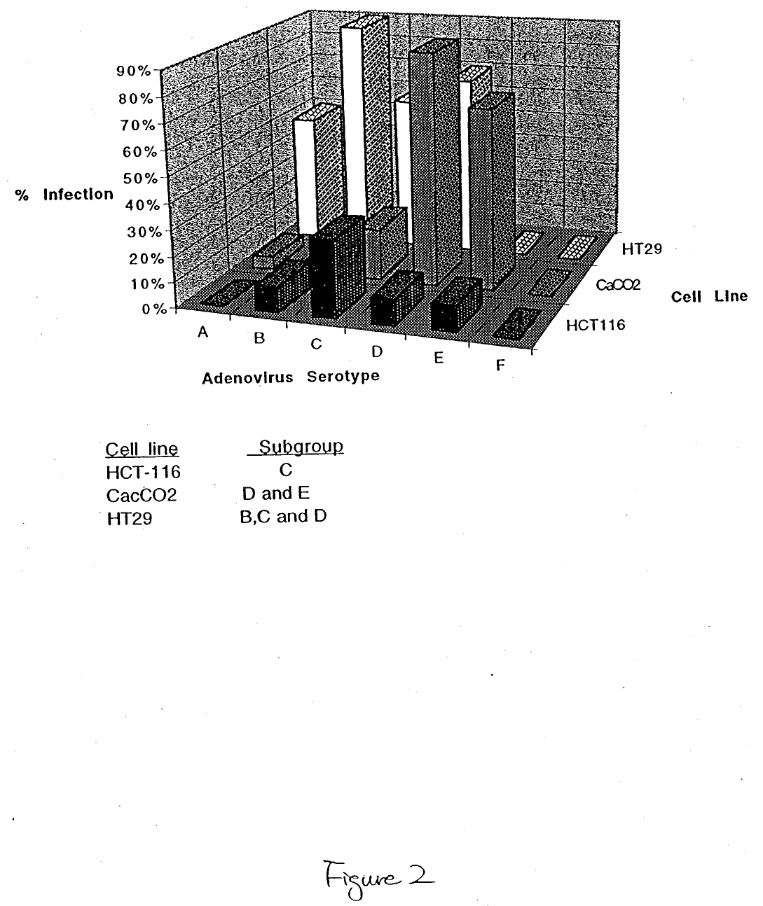

FIG. 2 shows infection efficiency of the various adenoviral subgroups into colon cancer cell lines, HCT116, CaCO2 and HT29. Subgroups D (Ad 17) and E (Ad 4) had a significantly higher infection rate of the CaCO2 cell line (infection rate>70%), while Ad 2 only infected 20% of the cells. The HCT cells were infected equally well by subgroups B (Ad 3) and C (Ad 2) at a rate of about 20%. The HT-29 cells were infected best by B (Ad 3), C (Ad 2) and D (Ad 17), at a rate of approximately 75%.

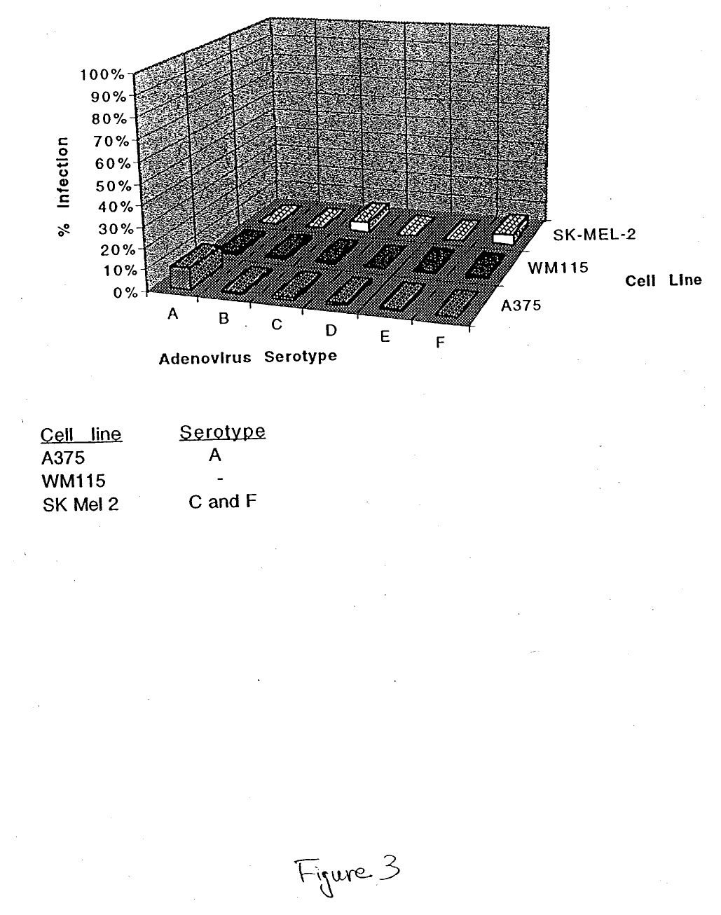

As shown in FIG. 3, the breast cancer cell line SK-BR3 was infected at higher rates by subgroups C (Ad 2), D (Ad 17) and E (Ad 4), while the ovarian cancer cell line SK-OV3 was infected best by subgroup D (Ad 17). HeLa cells, a cervical cancer cell line, were infected at higher rates by subgroup C (Ad 2), and prostate cancer cells (PC-3) by subgroups C (Ad 2) and E (Ad 4).

### Example 2

### Infection of Human Dendritic Cells With Different Adenovirus Serotypes

Following generally the procedures of Example 1, human dendritic cells and control A549 lung epithelial cells were infected with with a representative of each adenovirus subgroup at an moi of 50 and 5 respectively. Thirty-six hours post infection, cells were fixed and stained for hexon protein as described in Example 1.

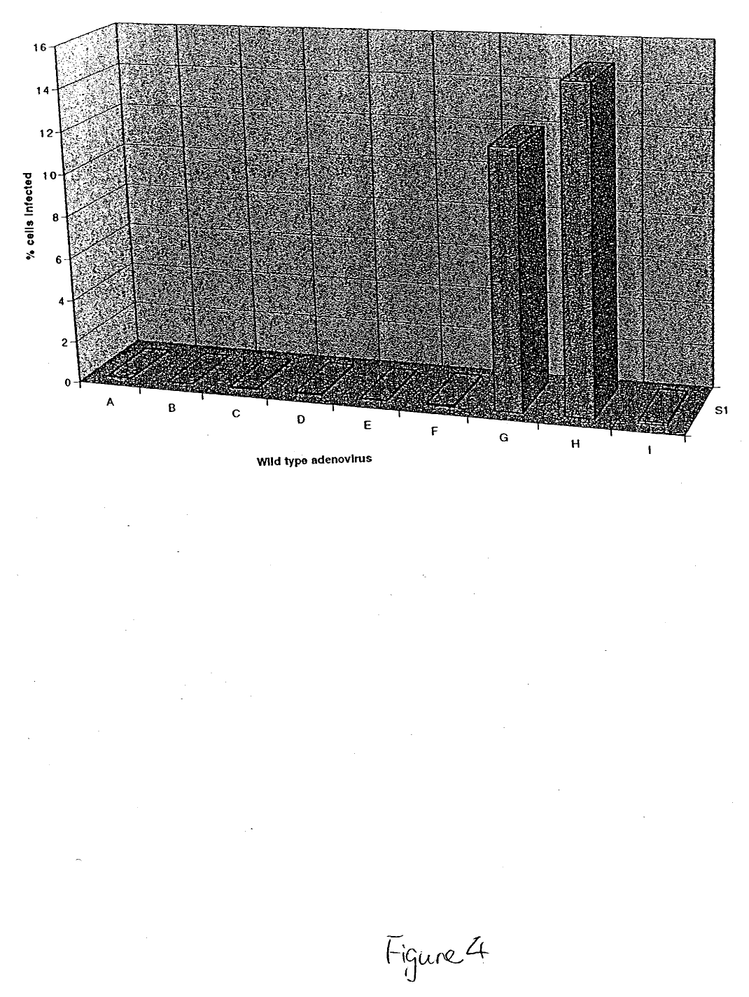

FIG. 4 shows that human dendritic cells were infected at a signicantly higher rate by subgroup D, particularly Ad 17 (depicted on plot of FIG. 4 as G) and Ad 19 (depicted on plot of FIG. 4 as H). Table I shows these results along with infection of the control cells. These results suggest that subgroup D viruses infect dendritic cells by at least 2-3 logs more efficiently than subgroup C viruses as measured by virus replication and hexon protein staining.

TABLE 1

| | | % cells stained positive | |
|---|---|---|---|
| Adenovirus | Subgroup | A549 (moi 5) | DC (moi 50) |
| Ad 31 (A)* | A | 25 | 1 cell |
| Ad 7 (B) | B | 100 | 0 |
| Ad 2 (C) | C | 100 | ˜4 cells |
| Ad 5 (D) | C | 100 | ˜5 cells |
| Ad 2 hexon/5 fiber (E) | C | 95 | ˜1 cell |
| Ad 5 hexon/2 fiber (F) | C | 100 | ˜1 cell |
| Ad 17 (G) | D | 100 | ˜12% |
| Ad 19 (H) | D | 100 | ˜15% |
| Ad 4(I) | B | 100 | ˜10 cells |

*Letter in brackets identifies the serotype in the plot of FIG. 4.

### Example 3

### Construction of Chimeric Adenoviral Vectors

In order to assess transgene expression and infection efficiency using different chimeric adenoviral vectors, lacZ gene encoding β-galactosidase was used as a transgene marker. The vector Ad2/βgal-2 was constructed as follows. A CMVβgal expression cassette was constructed in a pBR322-based plasmid that contained Ad2 nucleotides 1-10,680 from which nucleotides 357-3328 were deleted. The deleted sequences were replaced with (reading from 5′ to 3′): a cytomegalovirus immediate early promoter (obtained from pRC/CMV, Invitrogen), lacZ gene encoding β-galactosidase with a nuclear localization signal, and an SV40 polyadenylation signal (nucleotides 2533-2729). The resulting plasmid was used to generate Ad2/βgal-2 by recombination with Ad2E4ORF6 (Armentano et al. (1995) Human Gene Therapy 6:1343-1353).

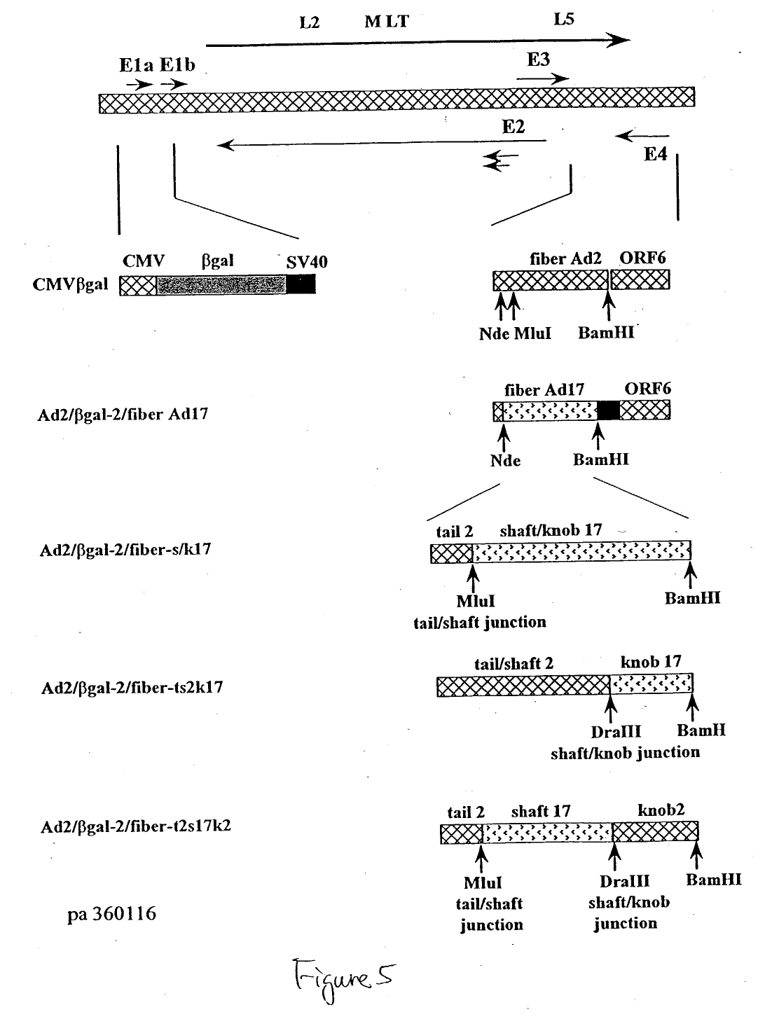

The following are different chimeric adenoviral vectors constructed according to the invention. A schematic map for each vector construct is shown in FIG. 5.

1. Ad2/βgal-2/fiber Ad17

The vector Ad 2/βgal-2/fiber Ad 17 was constructed as follows. PAdORF6 (Armentano, et al. (1995) Human Gene Therapy 6:1343-53) was cut with Nde and BamH1 to remove Ad 2 fiber coding and polyadenylation signal sequences (31078-32815). An NdeI-BamH1 fragment containing Ad 17 fiber coding sequence (31001-32053) was generated by PCR and ligated along with an SV40 polyadenylation signal into NdeI-BamH1 cut pAdORF6 to generate pAdORF6fiber17. This plasmid was cut with PacI and ligated to PacI-cut Ad 2/βgal-2 genomic DNA. The ligation was transfected into 293 cells, plaques were picked and virus was expanded and analyzed by restriction endonuclease digestion. The resulting virus contained the N-terminal 16 amino acids of the tail region from Ad 2 and the remainder of the tail, shaft and knob from Ad17.

2. Ad2/βgal-2/fiber-s/k17

Another chimeric vector, Ad 2/βgal-2/fiber-s/k17, was constructed that contains the entire tail region from Ad 2 and the shaft and knob region from Ad 17. This was done to improve yield and growth properties of an Ad 2/Ad 17 chimeric virus. Since the tail region of fiber interacts with penton base, it was assumed that maintaining the tail region from Ad 2 would lead to better fiber/penton base interaction and thus improve growth characteristics of the chimerics virus. PAdORF6 was cut with MluI and BamH1 to remove Ad 2 fiber shaft and knob coding and polyadenylation signal sequences (31177-32815). An MluI-BamHI fragment containing Ad 17 fiber shaft and knob coding sequence (31101-32053) was generated by PCR and ligated along with an SV40 polyadenylation signal to generate pAdORF6 fiber-s/k17. Chimeric viruses can be generated as described above.

3. Ad2/βgal-2/fiber-t2s17k2

Chimeric vector comprises the tail of Ad2, shaft of Ad17 and knob of Ad2. The starting construct was PAdORF6SVpae, which is identical to PAdORF6 except that it contains an SV40 polyadenylation signal between the fiber and ORF6 regions. PAdORF6SVpae was cut with MluI and BamHI to remove Ad2 fiber shaft and knob coding and polyadenylation signal sequences. The sequences were replaced with an MluI-DraIII fragment (31083-31488) of Ad 17 fiber shaft and a DraIII-BamHI fragment (32229-32815) of Ad2 fiber knob and polyadenylation signal sequences, both were generated by PCR, to generate pAdORF6fiber-t2s17k2. A virus containing this version of fiber was generated as described above. The shaft of Ad17 is considered shorter than that of Ad2. It is suggested that viruses with shortened shaft regions interact better with cell surface integrins via penton base, thus facilitating viral infection. The chimeric virus can be tested on a variety of cell lines for increased transduction capacity.

4. Ad2/βgal-2/fiber-ts2k17

Another chimeric vector can be generated by replacing the knob of Ad2 with that of Ad17. PAdORF6SVpae was cut with MluI and BamHI. PCR-generated fragments containing the Ad2 shaft as an MluI-DraIII fragment (31176-32232) and the Ad 17 knob and polyadenylation signal sequences as a DraIII-BamHI fragment (31493-32094) were added to generate pAdORF6fiber-ts2k17. A chimeric virus can be generated as described above.

### Example 4

### Infection of Human Dendritic Cells With Chimeric Adenovirus Vectors

A panel of different Adβgal vectors were used to determine if any of the fiber modified or hexon modified vector infect DCs more efficiently than Ad 2 or Ad 5 vectors.

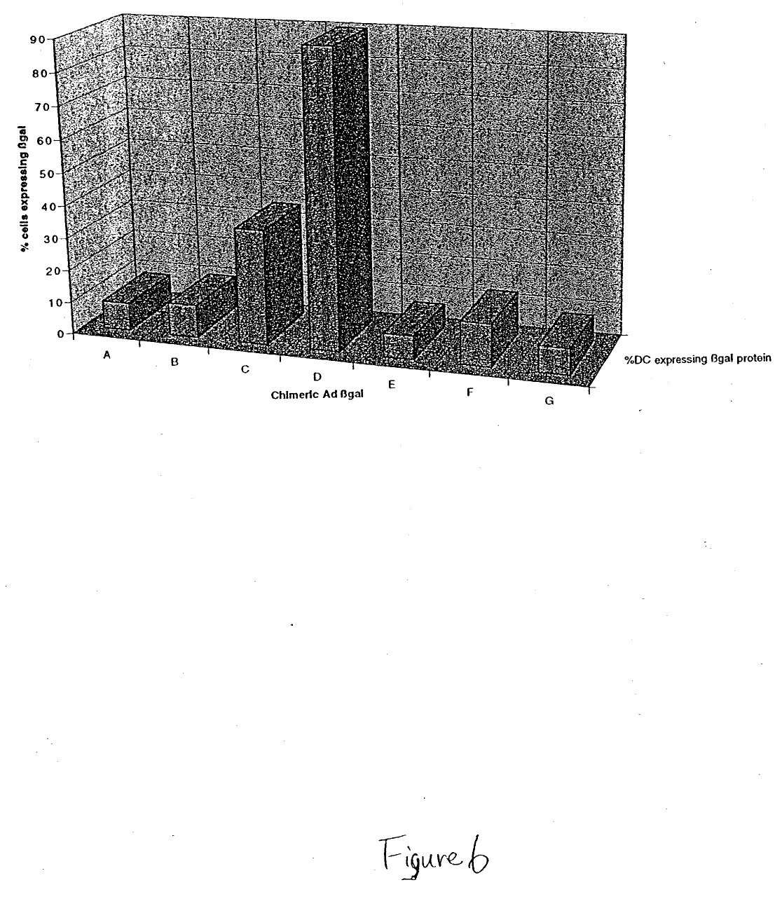

Human dendritic cells and A549 controls cells were seeded into a 96 well plate and infected with an moi of 50 and 5 respectively with a panel of Ad/βgal vectors shown in Table 2. Thirty six hours post-infection, cells were fixed and stained with X-gal and the percentage of positively stained cells were calculated for each βgal vector and for both DC and A549 cells. Table 2 and FIG. 6 show the percentage of DCs transduced when normalized to 100% A549 cells transduced for each vector.

TABLE 2

| | % DC expressing βgal protein as |
|---|---|
| Ad/βgal vector | measured by X gal staining |
| Ad2/βgal4 (A)* | ˜8.5 |
| Ad2ΔCMV/βgal (B) | ˜10 |
| Ad5/βgal (C) | ˜35 |
| Ad2/βgal/F17 (D) | ˜90 |
| Ad2/βgal 5RDG HM (E) | ˜7.1 |
| Ad2/βgal 17RGD HM (F) | ˜13 |
| Ad2/βgal SV40nls HM (G) | ˜8 |

*letter in brackets identifies the corresponding vectors in the plot of FIG. 6.

Of all the vectors tested, the chimeric adenoviral vector Ad2/βgal F17 gives rise to the significantly highest frequency of infected DC cells. All other vectors gave more or less the same percentage of transduced cells, indicating that the chimeric Ad vector with Ad17 fiber sequences infects DC very efficiently.

It is to be understood that while the invention has been described in conjunction with the above embodiments, that the foregoing description and examples are intended to illustrate and not limit the scope of the invention. For example, any of the above-noted compositions and/or methods can be combined with known therapies or compositions. Other aspects, advantages and modification within the scope of the invention will be apparent to those skilled in the art to which the invention pertains.

What is claimed is:

1. A chimeric adenoviral vector comprising the genome of a first adenovirus, wherein at least a portion of the nucleotide sequence encoding a protein that facilitates binding of the first adenovirus to a mammalian target cell is replaced by the corresponding nucleotide sequence from a second adenovirus, wherein the first and second adenoviruses are different serotypes.

2. A recombinant adenovirus comprising polypeptides encoded by the chimeric adenoviral vector of claim 1.

3. The chimeric adenoviral vector according to claim 1, wherein the corresponding nucleotide sequence from the second adenovirus encodes at least a portion of a fiber protein.

4. The chimeric adenoviral vector according to claim 3, wherein the portion of the fiber protein is selected from the group consisting of tail, shaft, knob and combinations thereof.

5. The chimeric adenoviral vector according to claim 1, wherein the corresponding nucleotide sequence from the second adenovirus encodes at least a portion of a penton base protein.

6. The chimeric adenoviral vector according to claim 1, wherein the corresponding nucleotide sequence from the second adenovirus encodes at least a portion of a hexon protein.

7. The chimeric adenoviral vector according to claim 1, wherein the first and second adenoviruses are of different serotype subgroups.

8. The chimeric adenoviral vector according to claim 7, wherein the first adenovirus is a subgroup C serotype and the second adenovirus is a subgroup A serotype, a subgroup B serotype, a subgroup D serotype, a subgroup E serotype or a subgroup F serotype.

9. The chimeric adenoviral vector according to claim 1, wherein the first adenovirus is Ad2 and the second adenovirus is Ad17.

10. The chimeric adenoviral vector according to claim 1, wherein the chimeric adenoviral vector is selected from the group consisting of Ad2/βgal-2/fiber Ad17, Ad2/βgal-2/fiber-s/k17, Ad2/βgal-2/fiber-t2s17k2, and Ad2/βgal-2/fiber-ts2k17.

11. The chimeric adenoviral vector according to claim 1, wherein the first adenovirus is replication-deficient.

12. The chimeric adenoviral vector according to claim 1, further comprising a transgene.

13. The chimeric adenoviral vector according to claim 12, wherein the transgene is a polynucleotide encoding for a tumor antigen, a costimulatory molecule or a cytokine.

14. The chimeric adenoviral vector according to claim 1, wherein the mammalian target cell is a melanoma cell and the second adenovirus is a subgroup C serotype, a subgroup A serotype, or a subgroup F serotype.

15. The chimeric adenoviral vector according to claim 1, wherein the mammalian target cell is a colon cancer cell and the second adenovirus is a subgroup B serotype, a subgroup C serotype, a subgroup D serotype, or a subgroup E serotype.

16. The chimeric adenoviral vector according to claim 1, wherein the mammalian target cell is a breast cancer cell and the second adenovirus is a subgroup C serotype, a subgroup D serotype, or a subgroup E serotype.

17. The chimeric adenoviral vector according to claim 1, wherein the mammalian target cell is an ovarian cancer cell and the second adenovirus is a subgroup D serotype.

18. The chimeric adenoviral vector according to claim 1, wherein the mammalian target cell is a cervical cancer cell and the second adenovirus is subgroup C serotype.

19. The chimeric adenoviral vector according to claim 1 wherein the mammalian target cell is a prostate cancer cell and the second adenovirus is a subgroup C serotype or subgroup E serotype.

20. The chimeric adenoviral vector according to claim 1 wherein the mammalian target cell is a dendritic cell and the second adenovirus is a subgroup D serotype.

21. A composition comprising a chimeric adenoviral vector according to claim 1 or 12 and a carrier.

22. A method of preferentially infecting a target mammalian cell comprising contacting the cell with a chimeric adenoviral vector according to claim 1.

23. A method of introducing a transgene to a target mammalian cell comprising infecting the cell with a chimeric adenoviral vector according to claim 12.

24. The method according to claim 22 or 23, wherein the target mammalian cell is a dendritic cell and the second adenovirus of the chimeric adenoviral vector is a subgroup D serotype.

25. The method according to claim 22 or 23, wherein the target mammalian cell is a melanoma cell and the second adenovirus of the chimeric adenoviral vector is a subgroup C serotype, a subgroup A serotype or a subgroup F serotype.

26. The method according to claim 22 or 23, wherein the target mammalian cell is a colon cancer cell and the second adenovirus of the chimeric adenoviral vector is a subgroup B serotype, a subgroup C serotype, a subgroup D serotype or a subgroup E serotype.

27. The method according to claim 22 or 23, wherein the target mammalian cell is a breast cancer cell and the second adenovirus of the chimeric adenoviral vector is a subgroup C serotype, a subgroup D serotype or a subgroup E serotype.

28. The method according to claim 22 or 23, wherein the target mammalian cell is an ovarian cancer cell and the second adenovirus of the chimeric adenoviral vector is a subgroup D serotype.

29. The method according to claim 22 or 23, wherein the target mammalian cell is a cervical cancer cell and the second adenovirus of the chimeric adenoviral vector is a subgroup C serotype.

30. The method according to claim 22 or 23, wherein the target mammalian cell is a prostate cancer cell and the second adenovirus of the chimeric adenoviral vector is a subgroup C serotype or a subgroup E serotype.

31. The method according to claim 23, wherein the cell is infected in vivo.

32. A mammalian cell comprising a transgene introduced by the method of claim 23.

33. The mammalian cell according to claim 32, wherein the transgene is a polynucleotide encoding for a tumor antigen, a costimulatory molecule or a cytokine.

34. The mammalian cell according to claim 33, wherein the mammalian cell is a dendritic cell.

35. A method of inducing an immune response in a subject comprising administering an effective population of the dendritic cells of claim 34 to the subject.

36. A method of inducing an immune response in a subject comprising administering an effective amount of the chimeric adenoviral vector of claim 13 to the subject.

37. A method of determining the infection efficiency of a known viral serotype to a target cell population, comprising the steps of:
a) incubating a virus of the known viral serotype with a predetermined cell population under the conditions such that the virus infects at least a portion of the target cell population; and
b) measuring the percentage of the target cell population being infected by the virus, thereby identifying the infection efficiency.

38. The method of claim 37, wherein the step (b) comprises staining of the target cell population with DAPI.

39. The method of claim 37, wherein the step (b) comprises staining of the target cell population with an antibody specific to the virus.

40. A recombinant adenovirus comprising adenoviral proteins encoded by the genome of a first adenovirus, wherein at least a portion of a protein that facilitates viral binding to a mammalian target cell is replaced by the corresponding protein portion from a second adenovirus, wherein the first and second adenoviruses have different serotypical phenotypes.

41. The recombinant adenovirus of claim 40, wherein the second adenovirus has preferred infection efficiency to the mammalian target cell.

42. The recombinant adenovirus of claim 40, wherein the protein that facilitates viral binding to the mammalian target cell is an adenoviral fiber protein.

43. The recombinant adenovirus of claim 40, wherein the protein that facilitates viral binding to the mammalian target cell is an adenoviral penton base protein.

44. The recombinant adenovirus of claim 40, wherein the protein that facilitates viral binding to the mammalian target cell is an adenoviral hexon protein.

45. The recombinant adenovirus of claim 40, further comprising a transgene product.

46. A method of preferentially infecting a target mammalian cell comprising contacting the cell with the recombinant adenovirus of claim 40.

47. A method of introducing a transgene product to a target mammalian cell comprising infecting the cell with the recombinant adenovirus of claim 45.

48. A gene expression system comprising a first adenovirus lacking a polypeptide that facilitates viral binding to a cell; and a packaging cell line that produces a polypeptide of a second adenovirus, wherein the activity of the polypeptide of the second adenovirus complements the phenotype of the first adenovirus, wherein the first and second adenoviruses are of different serotypes.

49. A method of characterizing an unknown type of a cell, comprising:
(a) determining a infection profile of the unknown cell type comprising the identities of the adenoviral subgroups that preferentially infect the unknown cell type; and
(b) comparing the infection from step (a) to infection profiles of known cells, thereby characterizing the unknown cell type.
